(19) United States
(12) Patent Application Publication
(10) Pub. No.: US 2008/0254029 A1
(43) Pub. Date: Oct. 16, 2008

(54) Use of an Inhibitor of TNFa Plus an Antihistamine to Treat Allergic Rhinitis and Allergic Conjunctivitis

(75) Inventors: John M. Yanni, Burleson, TX (US); Daniel A. Gamache, Arlington, TX (US); Steven T. Miller, Arlington, TX (US); Clay Beauregard, Fort Worth, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/100,715

(22) Filed: Apr. 10, 2008

Related U.S. Application Data
(60) Provisional application No. 60/911,176, filed on Apr. 11, 2007.

(51) Int. Cl. A61K 39/395; A61K 31/551; A61K 31/335; A61K 31/55; A61P 37/08; A61K 38/16; A61K 31/4353; A61K 31/52

(52) U.S. Cl. 424/133.1; 514/218; 514/450; 514/214.02; 514/291; 514/263.22; 424/158.1; 424/145.1; 514/12

(57) ABSTRACT

Disclosed are methods of treating allergic conjunctivitis and allergic rhinitis in a subject that involve topically administering to the subject a composition comprising a pharmaceutically effective amount of an H₁ antagonist and an anti-TNFα compound.

This application claims priority to U.S. Provisional Application, U.S. Ser. No. 60/911,176 filed Apr. 11, 2007.

### BACKGROUND OF THE INVENTION

1. Field of the Invention

The present invention relates generally to the field of inhibitors of tumor necrosis factor α (TNFα), antihistamines, pharmaceutics, and the treatment of allergic conjunctivitis and allergic rhinitis. More particularly, the present invention concerns methods of treating or preventing allergic conjunctivitis and allergic rhinitis in a subject that involve topically administering a composition comprising a pharmaceutically effective amount of an anti-TNFα agent and an anti-histaminic agent.

2. Description of Related Art

In industrialized countries, more than 10-15% of the population suffers from allergic rhinitis and/or conjunctivitis. Allergic rhinitis and/or conjunctivitis are type I allergic responses that are mediated by IgE antibodies. As a part of an allergic response to antigen, IgE is generated, which binds to the surface of mast cells and basophils via high affinity Fc receptors that are specific for IgE. Antigen cross-linking the IgE-molecules leads to cellular responses involving release of preformed mediators (e.g., histamine), lipid mediator formation and release, and cytokine generation. Mast cells with their mediators can be regarded as central to the initiation and mediation of allergic inflammation.

Clinical symptoms of allergic rhinitis include sneezing, nasal congestion, nasal itching, and rhinorrhea. Clinical symptoms of allergic conjunctivitis include watery discharge, redness, and edema of the eyelids. These symptoms may vary in intensity from the nuisance level to debilitating.

Allergic rhinitis often coexists with allergic conjunctivitis, and other disorders or conditions, such as asthma, sinusitis, atopic dermatitis, and the presence of nasal polyps. All these can frequently lead to significant impairment of quality of life.

Histamine has been implicated in allergic rhinitis and allergic conjunctivitis. Histamine is an important mediator released from mast cells that populate the walls of the nasal mucous membrane. When released, histamine is known to bind competitively to local histamine H₁ receptors and cause sneezing, nasal itching, and swelling of the nasal membranes. The primary action of antihistamines relates to their ability to bind competitively to H₁ histamine receptors on target organ sites, thereby blocking the ability of histamine to bind to these receptors. Anti-histamine compounds that bind to histamine receptors have been found to be useful in treating the signs and symptoms of these conditions. Most of these drugs are compounds that are structurally related to histamine and bind to its receptor(s), thereby obstructing the interaction of histamine with its receptor(s).

Conventional H₁ receptor antagonists (“H₁ antagonists”) are widely used as antihistamine agents for treating allergic conjunctivitis and allergic rhinitis. H₁ antagonists target some of the signs and symptoms including itching, sneezing, and inflammation that are associated with these conditions. One limitation of H₁ receptor antagonists is that they are antihistaminic only, providing primarily short-term relief of symptoms.

Other therapies for allergic rhinitis include leukotriene receptor antagonists, decongestants, nasal corticosteroids, intranasal antihistamines, intranasal cromolyns, and intranasal anticholinergic agents. These therapies have disadvantages, however, including steroid-related side effects (nasal corticosteroids), and absence of a direct anti-histaminic effect (intranasal cromolyns, leukotriene antagonists, and intranasal anticholinergic agents).

Tumor Necrosis Factor α (TNFα) is a cytokine that has been shown to play a pivotal role in immune and inflammatory responses, including allergic rhinitis and conjunctivitis. TNFα is a soluble homotrimer of 17 kD protein subunits (Smith, 1987). TNFα is derived from mononuclear cells and macrophages, along with other cell types. Modulation of TNFα has been proposed as a therapeutic strategy for allergic conjunctivitis, and other conditions associated with activation of TNFα.

The widespread incidence of allergic conjunctivitis and allergic rhinitis means that there is a continuing need for the discovery of therapies that are effective to ameliorate the signs and symptoms of this condition.

### SUMMARY OF THE INVENTION

The present invention overcomes drawbacks of the prior art by providing for novel formulations and methods for treating allergic conjunctivitis and allergic rhinitis. In particular, the inventors have found that treatment of allergic rhinitis or allergic conjunctivitis with a combination of an H₁ antagonist and an anti-TNFα compound provides both immediate and long-term relief.

The allergic conjunctivitis may be seasonal allergic conjunctivitis, perennial allergic conjunctivitis, vernal conjunctivitis, giant papillary conjunctivitis, or atopic keratoconjunctivitis.

In particular embodiments, the disease to be treated or prevented is allergic conjunctivitis, and administration is topical to the surface of an eye or periocular skin of the eyelids of the subject. In other particular embodiments, the disease to be treated or prevented is allergic rhinitis, and the therapeutic agents are administered topically into the nose, such as by drop or aerosol.

Although a wide variety of treatments for allergic rhinitis and allergic conjunctivitis are available, many have significant limitations or side effects. For example, anti-histamine products are anti-histaminic only and do not address the inflammation component of an allergic response, while nasal corticosteroids are associated with steroid side effects. There is a need for more effective therapies for allergic conjunctivitis and allergic rhinitis.

The inventors have identified novel methods for treating or preventing allergic conjunctivitis or allergic rhinitis in a subject that involve administering to the subject a pharmaceutically effective amount of a composition that includes an antihistamine and an anti-TNFα compound.

Although not wishing to be bound to any theory, it is believed that the combination of H₁ antagonist and anti-TNFα compound provides immediate relief from acute allergy effects such as sneezing, edema, nasal itching and rhinorrhea because of the H₁ antagonist and protection from allergic inflammation and congestion because of the anti-TNFα compound. The combination product of the present invention is devoid of the risk of steroid-induced side effects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

As used herein the specification, “a” or “an” may mean one or more. As used herein in the claim(s), when used in conjunction with the word “comprising”, the words “a” or “an” may mean one or more than one. As used herein “another” may mean at least a second or more.

### A. H₁ Antagonists

When released, histamine is known to bind competitively to local histamine H₁ receptors and cause sneezing, nasal itching, and swelling of the nasal membranes. A “first generation antihistamine” refers to an agent that binds competitively to H₁ histamine receptors on target organ sites, thereby blocking the ability of histamine to bind to these receptors. Examples of these so-called first-generation antihistamines include brompheniramine, diphenhydramine, promethazine, and hydroxyzine.

First generation antihistamines have been proven efficacious for preventing and relieving sneezing, itching, and other symptoms of the early allergic response, but have not been found to be very effective for relief of the nasal congestion which is a typical symptom of allergic rhinitis.

The sedating side effects of antihistamines have stimulated the development and marketing of the so-called second generation antihistamines. Examples include loratadine, cetirizine, terfenadine, astemizole, azelastine, fexofenadine. These agents are less lipophilic than the first generation antihistamines, conferring a reduction in their ability to cross the blood-brain barrier and thereby cause sedation. Some of these second-generation antihistamines have a concomitant diminution of anticholinergic effects and thus decreased potency for controlling rhinorrhea.

Third generation of antihistamines include agents that are either metabolites or isomers of second generation antihistamines. Examples include desloratadine and levocetirizine. Their advantage compared to second generation antihistamines is seen in an improved safety profile and decreased antimuscarinic/anticholinergic effects.

The topical (nasal) histamine H₁-receptor antagonists, azelastine, levocabastine, and dimetinden are an established anti-rhinitis therapy. Azelastine is a pharmacologically distinct histamine H₁-receptor antagonist with a broad spectrum of antiallergic activity. Azelastine and levocabastine are available worldwide as nasal spray formulations and approved for treatment of allergic rhinitis; in the United States azelastine is also available to treat non-allergic vasomotor rhinitis.

Preferred H₁ receptor antagonists include cetirizine, azelastine, levocabastine, emedastine, olopatadine, epinastine, bepotastine, mizolastine, desloratadine, levocetirizine, and dimetinden. Most preferred are emedastine, epinastine, and olopatadine.

### B. Anti-TNF

An “anti-TNF compound” is defined herein to refer to an agent that decreases, blocks, inhibits, abrogates or interferes with TNF activity in vitro or in vivo. Thus, for example, an anti-TNFα is an agent that blocks, impairs, or inhibits the action of TNFα. For example, the anti-TNFα can be an inhibitor of the synthesis of TNFα (such as a PDE4 inhibitor, a JAK3 inhibitor, or a p38 kinase inhibitor), a TNFα antagonist (such as a small molecule), such as an agent that inhibits the binding of TNFα to a TNF receptor, an antibody (such as an anti-TNF antibody or an anti-TNF receptor antibody), or a TNFα sink (such as a soluble receptor). In general, an anti-TNFα can be a small molecule, a peptide, a protein, an antibody, a DNA, an RNA (such as an siRNA or mRNA), or an oligonucleotide. One of ordinary skill in the art would be familiar with this class of agents.

Tumor necrosis factor (TNF) is a cytokine produced by activated macrophages (TNF-α), mast cells and some T cells (TNF-β), which elicits a wide range of biological activities, including inflammatory, immunoregulatory, proliferative, cytotoxic and anti-viral activities. The term “human TNFα”, as used herein, is intended to refer to a human cytokine that exists as a 17 kD secreted form and a 26 kD membrane associated form, the biologically active form of which is composed of a trimer of noncovalently bound 17 kD molecules. The structure of human TNFα is described further in Pennica et al. (1984); Davis et al., (1987); and Jones et al., (1989).

For example, “anti-TNFα” includes agents that can bind TNFα such as anti-TNFα antibodies. Also included as anti-TNFα are receptor molecules which bind specifically to TNFα. Anti-TNFα also includes agents that can prevent or inhibit TNFα synthesis and/or TNFα release.

Particular TNFα antagonists for inclusion in the methods set forth herein include etanercept (sold as ENBREL® from Wyeth-Ayerst Laboratories/Immunex); infliximab (an anti-TNF chimeric Mab sold as REMICADE® from Centocor); D2E7 human Mab (Cambridge Antibody Technology); adalimumab (sold as HUMIRA® by Abbott), CDP-870, CDP-571, Humicade, which is a humanized Mab described in U.S. Pat. No. 5,994,510 (Celltech); PEGylated soluble TNFα Receptor-1 (Amgen); TBP-1, which is a TNF binding protein (Ares Serono); PASSTNF-alpha®, which is an anti-TNFα polyclonal antibody (Verigen); ienercept, which is a TNFR-Ig fusion protein (sold as TENEFUSE® from Roche); CytoTAb® (Protherics); TACE, which is a small molecule TNFα converting enzyme inhibitor (Immunex); small molecule TNF mRNA synthesis inhibitor (Nereus); PEGylated p75 TNFR Fc mutein (Immunex); and TNFα antisense inhibitor.

One of ordinary skill in the art would be familiar with the class of agents that can be categorized as anti-TNFα. Additional details regarding examples of anti-TNFα are set forth as follows.

1. Inhibitors of the Synthesis of TNFα

a. PDE4 Inhibitors

The cyclic nucleotide specific phosphodiesterases (PDEs) represent a family of enzymes that catalyze the hydrolysis of various cyclic nucleoside monophosphates (including cAMP and cGMP). PDE enzymes can be grouped into eleven families according to their specificity toward hydrolysis of cAMP or cGMP, their sensitivity to regulation by calcium, calmodulin or cGMP, and their selective inhibition by various compounds. PDE 4 is cAMP specific, and its inhibition causes airway relaxation, anti-inflammatory and antidepressant activity. The PDE4 enzyme family consists of four genes, which produce 4 isoforms of the PDE4 enzyme designated PDE4A, PDE4B, PDE4C, and PDE4D (Wang et al., 1997). Inhibitors of phosphodiesterases (PDEs) are a class of agents that inhibit the synthesis of TNFα.

PDE4 isoenzymes are localized in the cytosol of cells and are unassociated with any known membranous structures. PDE4 isoenzymes specifically inactivate cAMP by catalyzing its hydrolysis to adenosine 5′-monophosphate (AMP). Regulation of cAMP activity is important in many biological processes, including inflammation and memory.

Inhibitors of PDE4 isoenzymes such as rolipram, piclamilast, CDP-840 and ariflo are powerful anti-inflammatory agents that inhibit the synthesis of TNFα. In addition to such compounds as rolipram, xanthine derivatives such as pentoxifylline, denbufylline, and theophylline inhibit PDE4.

Additional inhibitors of PDE4 contemplated for inclusion by the methods set forth herein include pyridine N-oxide analogs of N-substituted diarylamine compounds (described in U.S. Pat. No. 7,087,625), substituted 8-arylquinoline phosphodiesterase-4 inhibitors (described in U.S. Pat. No. 6,740,666), alkyne-aryl phosphodiesterase-4 inhibitors (described in U.S. Pat. No. 6,743,802), 1-aryl-1,8-naphthyridin-4-one phosphodiesterase inhibitors (described in U.S. Pat. Nos. 6,677,351 and 6,541,480), hydroxyindoles (described in U.S. Patents RE38,624, 6,613,794 and 6,602,890), phthalazine derivatives (described in U.S. Pat. No. 6,589,951), tricyclic phthalazine derivatives (described in U.S. Pat. No. 6,525,055), benzazine derivatives (described in U.S. Pat. No. 6,358,973), benzamides with tetrahydrofuranyloxy substituents (U.S. Pat. No. 6,303,789), diazepinoindolones (described in U.S. Pat. No. 6,239,130), 1-oxo-1-3-substituted phenyl-1,4-dihydro-1,8-naphthyridine-3-carboxamide phosphodiesterase-4 inhibitors (described in U.S. Patent App. Pub. No. 20060058316), N-substituted diarylamines (described in U.S. Patent App. Pub. No. 20050222207), allyne-aryl phosphodiesterase-4 inhibitors (described in U.S. Patent App. Pub. No. 20050070569), and naphthyridine derivatives (described in U.S. Patent App. Pub. No. 20040254212). Each of the patents and patent applications set forth in this paragraph is herein specifically incorporated by reference in its entirety.

Additional inhibitors of PDE4 can be identified using any method known to those of ordinary skill in the art. Examples of such methods include those methods set forth in U.S. Pat. No. 6,909,002, and U.S. Patent App. Pub. No. 20060019981, each of which is herein incorporated by reference in its entirety.

b. JAK3 Inhibitors

Another class of agents that inhibit TNFα production include Janus kinase 3 (JAK3) inhibitors. JAK3 mediates signal transduction from cytokine receptors using the common chain (gammac). Mutations in genes encoding gammac or JAK3 result in immunodeficiency. Janus kinase 3 (Jak3) is a tyrosine kinase expressed in hematopoietic cells that associates with the common gamma chain and is required for signaling for a family of cytokines including interleukin-2 (IL-2), IL-4, IL-7, IL-9, IL-15, and IL-21; deficiency of either Jak3 or the gamma common chain results in severe combined immunodeficiency (SCID). JAK3 has been found to negatively regulate dendritic cell cytokine production and survival (Yamaoka et al., 2005).

Exemplary JAK3 inhibitors include tacrolimus, CP-690550, WHI-P131, WHIP-97, WHIP-154, AG490, PS-608504, and PNU156804. Additional exemplary JAK3 inhibitors include:

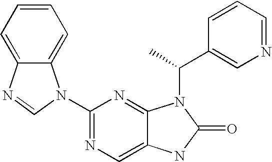

### 2-(1H-Benzimidazol-1-yl)-9-[1(R)-(3-pyridyl)ethyl]-8,9-dihydro-7H-purin-8-one;

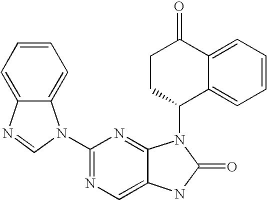

### 2-(1H-Benzimidazol-1-yl)-9-[4-oxo-1,2,3,4-tetrahydronaphthalen-1(R)-yl]-8,9-dihydro-7H-purin-8-one;

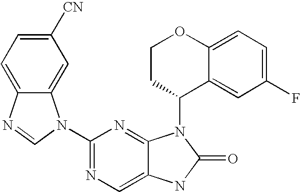

### 1-[9-[6-Fluoro-3,4-dihydro-2H-1-benzopyran-4(R)-yl]-8-oxo-8,9-dihydro-7H-purin-2-yl]-1H-benzimidazole-6-carbonitrile;

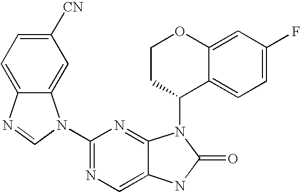

### 1-[9-[7-Fluoro-3,4-dihydro-2H-1-benzopyran-4(R)-yl]-8-oxo-8,9-dihydro-7H-purin-2-yl]-1H-benzimidazole-6-carbonitrile; and

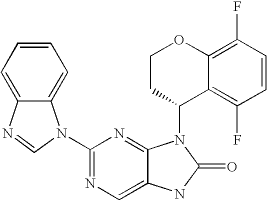

### 2-(1H-Benzimidazol-1-yl)-9-[5,8-difluoro-3,4-dihydro-2H-1-benzopyran-4(R)-yl]-8,9-dihydro-7H-purin-8-one

c. p38 Kinase Inhibitors

p38 kinase inhibitors are a known class of compounds. Suitable p38 kinase inhibitors include 3(5)-heteroaryl substituted pyrazoles (U.S. Pat. No. 5,932,425). Additional p38 kinase inhibitors include 1-(5-tert-butyl-2-p-tolyl-2H-pyrazol-3-yl)-3-[4-(2-morpholin-4-yl-ethoxy)naphthalen-1-yl]urea (BIRB 796); SB202190; SB203580; VX-745; and VX-702. Still other p38 kinase inhibitors include those disclosed in the following U.S. Pat. Nos. 7,189,731; 7,183,287; 7,173,129; 7,135,575; 7,067,540; 6,979,693; 6,696,471; 6,630,485; 6,579,874; 6,479,507; 6,444,696; and 6,316,464, the entire contents of which are each specifically incorporated by reference.

2. TNF Antagonists

A “TNF antagonist” is defined herein to refer to an agent that inhibits or impairs the binding of TNF to a TNF receptor. The agent can be, for example, a small molecule, a peptide, a protein, an antibody, a DNA, or an RNA.

a. Antibodies

In particular embodiments, the TNFα antagonist is an antibody. The term “antibody” is defined herein to include polyclonal antibodies, monoclonal antibodies (mAbs), chimeric antibodies, anti-idiotypic (anti-Id) antibodies to antibodies that can be labeled in soluble or bound form, as well as fragments, regions or derivatives thereof, provided by any known technique, such as, but not limited to, enzymatic cleavage, peptide synthesis or recombinant techniques. Anti-TNF antibodies include antibodies that are capable of binding portions of TNF or TNF receptors such that the binding of TNF to TNF receptors is inhibited. Anti-TNFα antibodies refers to antibodies that are capable of binding portions of TNFα to TNFα receptors such that the binding of TNFα to TNFα receptors is inhibited.

“Polyclonal antibodies” are defined herein to refer to heterogeneous populations of antibody molecules derived from the sera of animals immunized with an antigen. A “monoclonal antibody” contains a substantially homogeneous population of antibodies specific to antigens, which population contains substantially similar epitope binding sites. Mabs may be obtained by methods known to those skilled in the art. See, e.g., Kohler and Milstein, 1975; U.S. Pat. No. 4,376,110; Ausubel et al., 1992); Harlow and Lane 1988; Colligan et al., 1993, the contents of which are each herein specifically incorporated by reference. Such antibodies may be of any immunoglobulin class including IgG, IgM, IgE, IgA, GILD and any subclass thereof. A hybridoma producing a mAb of the present invention may be cultivated in vitro, in situ or in vivo. Production of high titers of mAbs in vivo or in situ makes this the presently preferred method of production.

“Chimeric antibodies” are molecules, different portions of which are derived from different animal species, such as those having variable region derived from a murine mAb and a human immunoglobulin constant region, which are primarily used to reduce immunogenicity in application and to increase yields in production. Chimeric antibodies and methods for their production are known in the art. Exemplary methods of production are described in Cabilly et al., 1984; Boulianne et al., 1984; and Neuberger et al., 1985, each of which are herein incorporated by reference in their entirety.

An “anti-idiotypic antibody” (anti-Id) is an antibody which recognizes unique determinants generally associated with the antigen-binding site of an antibody. An Id antibody can be prepared by immunizing an animal of the same species and genetic type (e.g., mouse strain) as the source of the mAb with the mAb to which an anti-Id is being prepared. The immunized animal will recognize and respond to the idiotypic determinants of the immunizing antibody by producing an antibody to these idiotypic determinants (the anti-Id antibody). An exemplary method of producing such antibodies is found in U.S. Pat. No. 4,699,880, which is herein entirely incorporated by reference.

Anti-TNF antibodies of the present invention can include at least one of a heavy chain constant region, a heavy chain variable region, a light chain variable region and a light chain constant region, wherein a polyclonal Ab, monoclonal Ab, fragment and/or regions thereof include at least one heavy chain variable region or light chain variable region that binds a portion of a TNF and inhibits and/or neutralizes at least one TNF biological activity.

Anti-TNF antibodies include high affinity human-murine chimeric anti-TNF antibodies, and fragments or regions thereof, that have potent inhibiting and/or neutralizing activity in vivo against human TNFα. Such antibodies and chimeric antibodies can include those generated by immunization using purified recombinant human TNFα or peptide fragments thereof.

Additional information regarding anti-TNF antibodies and methods of production of anti-TNF antibodies, including anti-TNFα antibodies, can be found in U.S. Patent App. Pub. No. 20060153846, U.S. Patent App. Pub. No. 20060140949, U.S. Patent App. Pub. No. 20060121037, U.S. Patent App. Pub. No. 20060024310, U.S. Patent App. Pub. No. 20060024308, U.S. Patent App. Pub. No. 20060018907, U.S. Patent App. Pub. No. 20050123541, U.S. Pat. No. 7,101,674, U.S. Pat. No. 7,060,800, U.S. Pat. No. 7,057,022, U.S. Pat. No. 6,991,791, U.S. Pat. No. 6,835,823, U.S. Pat. No. 6,790,444, U.S. Pat. No. 6,277,969, and U.S. Pat. No. 6,270,766, the entire contents of which are each specifically incorporated by reference.

The preferred antibodies are recombinant human antibodies. Most preferred are infliximab (the active ingredient in REMICADE® and adalimumab (the active ingredient in HUMIRA®).

b. Other TNFα Antagonists

Other TNFα Antagonists may act by interfering with the maturation of TNF. Metalloprotease inhibitors that inhibit the activity of TNF converting enzyme (TACE) have been reported to interfere with TNF maturation. Examples of these inhibitors are set forth in U.S. Pat. No. 5,872,146, herein incorporated by reference. U.S. Pat. Nos. 5,981,701 and 5,695,953, herein incorporated by reference, describe non-proteolytic peptides capable of interacting with TNF to inhibit the binding of TNF to cells.

TNFα Antagonists also include soluble TNF receptors that competitively inhibit binding of TNF to its cell bound receptor. For example, etanercept (sold as ENBREL® from Immunex Corporation, Seattle, Wash.) binds specifically with TNF and blocks it interaction with cell surface TNF receptors. The soluble, extracellular portions of both TNFR1 (p55) and TNFR2 (p75) naturally bind to TNFα and can be used, alone or bound to another molecule, as another TNFα antagonist set forth herein. TNFα antagonists that incorporate a soluble fragment of one or both of these receptors are set forth in U.S. Pat. Nos. 5,482,130 and 5,514,582, both of which are herein incorporated by reference.

TNFα antagonists also include compounds that inhibit TNF signaling. For example, these can include polypeptides that inhibit binding to the intracellular domain of TNF receptor and thus inhibit or modulate signal transduction by the receptor. These inhibitors are described in U.S. Pat. Nos. 5,948,638; 5,891,675; 5,852,173; 5,849,501; 5,843,675; 5,712,381; 5,563,039; 5,789,550; and 5,708,142, each of which is herein incorporated by reference.

Other suitable TNFα antagonists include agents that reduce the levels of TNFα in tissues, and include the compounds described in U.S. Pat. Nos. 5,994,620; 5,981,701; 5,594,106; 5,336,603 and 4,565,397, each of which is herein incorporated by reference.

### C. Treatment of Disease

1. Definitions

“Treatment” and “treating” refer to administration or application of a therapeutic agent to a subject or performance of a procedure or modality on a subject for the purpose of obtaining a therapeutic benefit of a disease or health-related condition. Treating includes inhibiting the state, disorder or condition, i.e., arresting or reducing the development of the disease or at least one clinical or subclinical symptom thereof, or relieving the disease, i.e., causing regression of the state, disorder or condition or at least one of its clinical or subclinical symptoms. The benefit to a subject to be treated is either statistically significant or at least perceptible to the patient or to the physician. For example, in the context of the present invention, allergic conjunctivitis may be treated by topically applying to the ocular surface a pharmaceutically effective amount of an anti-histamine and an anti-TNF to reduce itching, redness, and irritation of the conjunctiva.

The term “therapeutic benefit” or “therapeutically effective” as used throughout this application refers to anything that promotes or enhances the well-being of the subject with respect to the medical treatment of his condition. This includes, but is not limited to, a reduction in the frequency or severity of the signs or symptoms of a disease. For example, regarding the treatment of allergic rhinitis, a therapeutic benefit is obtained when there is decreased rhinorrhea.

A “pharmaceutically effective amount” means the amount of a compound that, when administered to a mammal for treating a state, disorder or condition, is sufficient to effect such treatment. The “pharmaceutically effective amount” will vary depending on the compound, the disease and its severity and the age, weight, physical condition and responsiveness of the mammal to be treated.

2. Diseases to be Treated

The methods set forth herein can be applied in the treatment of allergic conjunctivitis or allergic rhinitis. Conjunctivitis is an inflammatory disease that affects the conjunctiva of one or both eyes of an individual. Symptoms and signs include redness, tearing, discharge, irritation, and itching of the eyes. The allergic conjunctivitis may be seasonal allergic conjunctivitis, perennial allergic conjunctivitis, giant papillary conjunctivitis, atopic keratoconjunctivitis, or vernal conjunctivitis. Rhinitis is inflammation of the lining of the nose, which may be caused by allergies or other factors such as cigarette smoke, changes in temperature, exercise and stress. Symptoms include sneezing, nasal congestion, nasal itching, and rhinorrhea.

### D. Pharmaceutical Compositions and Routes of Administration

One embodiment of this invention includes methods of treating allergic conjunctivitis or allergic rhinitis by administering a pharmaceutically effective amount of a composition that includes an H₁ antagonist and an anti-TNFα compound to a subject. The administration is topical to the eye or nose. As used herein, administration topical to the eye includes topical compositions dropped or placed on the eye or placed underneath the eye lids, as well as compositions applied to the periocular skin and surface of the eyelids. As used herein, administration topical to the nose includes delivering compositions by drop or spray into the nostrils and nasal passages.

The amount of drug to be included in the compositions or applied in the methods set forth herein will be whatever amount is pharmaceutically effective and will depend upon a number of factors, including the identity and potency of the chosen drug. One of ordinary skill in the art would be familiar with factors that are involved in determining a pharmaceutically effective dose of a drug.

In particular embodiments, the total concentration of the therapeutic agent (anti-TNFα and/or antihistamine) is about 5% (w/v) or less in the formulation. In general, the concentration of the H₁ antagonist in the compositions of the present invention will be from 0.0001% to 0.5% (w/v), preferably from 0.01 to 0.2% (w/v), and most preferably from 0.05 to 0.2% (w/v), while the concentration of the anti-TNFα compound will be from 0.0001 to 5% (w/v), preferably from 0.001 to 1% (w/v), and most preferably from 0.01 to 0.5% (w/v).

1. Ophthalmic Formulations

In particular embodiments, the compositions are suitable for topical application to mammalian eyes. For example, for ophthalmic administration, the formulation may be a solution, a suspension, a gel, or an ointment. The compositions are preferably formulated for topical application to the eye in aqueous solution in the form of drops. The term “aqueous” typically denotes an aqueous composition wherein the carrier is to an extent of >50%, more preferably >75% and in particular >90% by weight water. These drops may be delivered from a single dose ampoule which may preferably be sterile and thus rendering bacteriostatic components of the formulation unnecessary. These drops may also be delivered from a multi-dose container, particularly when the composition contains a preservative ingredient. Alternatively, the drops may be delivered from a multi-dose bottle which may preferably comprise a device which extracts preservative from the formulation as it is delivered, such devices being known in the art.

In other aspects, components of the invention may be delivered to the eye as a concentrated gel or similar vehicle which forms dissolvable inserts that are placed beneath the eyelids.

In addition the components can be place onto the outer eye lid and periocualr skin in a skin cream, gel, ointment, or lotion formulation.

In addition to the active ingredients, the compositions of the present invention may contain excipients. For example, the compositions may include one or more pharmaceutically acceptable buffering agents, preservatives (including preservative adjuncts), tonicity-adjusting agents, surfactants, solubilizing agents, stabilizing agents, comfort-enhancing agents, polymers, emollients, pH-adjusting agents and/or lubricants.

Suitable buffering agents include phosphates, borates, citrates, acetates and the like. Examples of preservatives include quaternary ammonium compounds, such as benzalkonium chloride, benzododecinium bromide, or polyquaternium-1. Other examples of preservatives include sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, or sorbic acid. Suitable tonicity-adjusting agents include mannitol, sodium chloride, glycerin, sorbitol and the like. Suitable surfactants include ionic and nonionic surfactants, though nonionic surfactants are preferred, such as polysorbates, polyethoxylated castor oil derivatives and oxyethylated tertiary octylphenol formaldehyde polymer (tyloxapol). Suitable chelating agents include sodium edetate and the like. Suitable antioxidants include sulfites, ascorbates, BHA and BHT.

Topical ophthalmic compositions are preferably isotonic, or slightly hypotonic in order to combat any hypertonicity of tears caused by evaporation and/or disease. The compositions of the present invention generally have an osmolality in the range of 220-320 mOsm/kg, and preferably have an osmolality in the range of 235-260 mOsm/kg. The compositions of the invention have a pH in the range of 5-9, preferably 6.5-7.5, and most preferably 6.8-7.4.

In certain embodiments, the therapeutic agents are formulated in a composition that comprises one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. The formulation of the present invention may be used with contact lenses or other ophthalmic products.

2. Nasal Formulations

In particular embodiments, the compositions of the present invention are administered topically to the nose. Topical nasal compositions are known and include aerosols and aqueous sprays or mists. As in the case of ophthalmic compositions, nasal compositions may contain excipients. For example, the compositions may include one or more pharmaceutically acceptable buffering agents, preservatives (including preservative adjuncts), tonicity-adjusting agents, surfactants, solubilizing agents, stabilizing agents, comfort-enhancing agents, polymers, emollients, pH-adjusting agents and/or lubricants.

### E. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Topical Ophthalmic Composition

| Ingredient | Amount (% w/v) |
|---|---|
| Emedastine | 0.05 |
| Etanercept | 5.0 |
| Dibasic Sodium Phosphate or | 0.01-0.5 |
| Tromethamine | |
| Sodium Chloride | 0.1-0.8 |
| Mannitol or Sucrose | 1-5 |
| Polysorbate 80 | 0.01-0.5 |
| NaOH and/or HCl | Adjust pH 7 ± 2 |
| Polyquad* | 0-0.005 |
| Purified Water | Q.S. 100 |

*If composition will be packaged for multi-dose use.

### Example 2

### Topical Nasal Composition

| Ingredient | Amount (% w/v) |
|---|---|
| Olopatadine | 0.1 |
| Adalimumab | 5.0 |
| Dibasic Sodium Phosphate | 0.01-0.5 |
| Sodium Chloride | 0.1-0.8 |
| Mannitol or Sucrose | 1-5 |
| Polysorbate 80 | 0.01-0.5 |
| NaOH and/or HCl | Adjust pH 5.5 ± 2 |
| Benzalkonium Chloride* | 0-0.02 |
| Purified Water | Q.S. 100 |

*If composition will be packaged for multi-dose use

### Example 3

### Ointment Composition

| Ingredient | Amount (% w/v) |
|---|---|
| Olopatadine | 0.1 |
| Etanercept | 5.0 |
| Mineral Oil | 0.1-0.5 |
| Petrolatum | Q.S.100 |

### Example 4

### Lotion Composition

| Ingredient | Amount (% w/v) |
|---|---|
| Olopatadine | 0.1 |
| Etanercept | 5.0 |
| Cetyl Alcohol | 2.6 |
| Stearyl Alcohol | 0.26 |
| Propyl Paraben | 0.05 |
| Methyl Paraben | 0.1 |
| Propylene Glycol | 2.0 |
| Sodium Lauryl Sulfate | 0.25 |
| NaOH/HCl | Q.S. to pH 5.5-7.5 |
| Purified Water | Q.S. 100 |

### Example 5

### Aqueous Gel Composition

| Ingredient | Amount (% w/v) |
|---|---|
| Olopatadine | 0.1 |
| Etanercept | 5.0 |
| Carbomer | 0.2-0.5 |
| Benzalkonium Chloride | 0.01 |
| Mannitol | 4.0 |
| NaOH/HCl | Q.S. to pH 5.5-7.5 |
| Purified Water | Q.S. 100 |

All of the methods disclosed and claimed herein can be executed without undue experimentation in light of the present disclosure. While the methods of this invention have been described in terms of preferred embodiments, it will be apparent to those of skill in the art that variations may be applied in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for treating allergic conjunctivitis or allergic rhinitis in a human subject, comprising topically administering to the eye or nose of the subject a composition comprising a pharmaceutically effective amount of an H₁ antagonist and a pharmaceutically effective amount of an anti-TNFα compound.

2. The method of claim 1, wherein the allergic conjunctivitis is selected from the group consisting of seasonal allergic conjunctivitis; perennial allergic conjunctivitis;
giant papillary conjunctivitis; vernal conjunctivitis; and atopic keratoconjunctivitis.

3. The method of claim 1, wherein the H₁ antagonist is selected from the group consisting of cetirizine; azelastine; levocabastine; emedastine; olopatadine;
epinastine; bepotastine; mizolastine; desloratadine; levocetirizine; and dimetinden

4. The method of claim 1 wherein the H₁ antagonist is selected from the group consisting of emedastine; olopatadine; and epinastine.

5. The method of claim 1, wherein the anti-TNFα compound is selected from the group consisting of inhibitors of TNFα synthesis and TNFα antagonists.

6. The method of claim 5 wherein the anti-TNFα compound is selected from the group consisting of inhibitors of TNFα synthesis.

7. The method of claim 6 wherein the anti-TNFα compound is selected from the group consisting of PDE4 inhibitors; JAK3 inhibitors; and p38 kinase inhibitors.

8. The method of claim 7 wherein the anti-TNFα compound is selected from the group consisting of PDE4 inhibitors.

9. The method of claim 7 wherein the anti-TNFα compound is selected from the group consisting of JAK3 inhibitors.

10. The method of claim 9 wherein the anti-TNFα compound is selected from the group consisting of tacrolimus; CP-690550; WHI-P131; WHIP-97; WHIP-154; AG490; PS-608504; PNU156804; 2-(1H-Benzimidazol-1-yl)-9-[1(R)-(3-pyridyl)ethyl]-8,9-dihydro-7H-purin-8-one; 2-(1H-Benzimidazol-1-yl)-9-[4-oxo-1,2,3,4-tetrahydronaphthalen-1(R)-yl]-8,9-dihydro-7H-purin-8-one; 1-[9-[6-Fluoro-3,4-dihydro-2H-1-benzopyran-4(R)-yl]-8-oxo-8,9-dihydro-7H-purin-2-yl]-1H-benzimidazole-6-carbonitrile; 1-[9-[7-Fluoro-3,4-dihydro-2H-1-benzopyran-4(R)-yl]-8-oxo-8,9-dihydro-7H-purin-2-yl]-1H-benzimidazole-6-carbonitrile; and 2-(1H-Benzimidazol-1-yl)-9-[5,8-difluoro-3,4-dihydro-2H-1-benzopyran-4(R)-yl]-8,9-dihydro-7H-purin-8-one.

11. The method of claim 7 wherein the anti-TNFα compound is selected from the group consisting of p38 kinase inhibitors.

12. The method of claim 5 wherein the anti-TNFα compound is selected from the group consisting of TNFα antagonists.

13. The method of claim 12 wherein the anti-TNFα compound is selected from the group consisting of anti-TNFα antibodies.

14. The method of claim 13 wherein the anti-TNFα compound is selected from the group consisting of infliximab and adalimumab.

15. The method of claim 1 wherein the anti-TNFα compound is etanercept.

16. The method of claim 1 wherein the anti-TNFα compound is selected from the group consisting of etanercept; infliximab; and adalimumab.

17. The method of claim 1 wherein the pharmaceutically effective amount of the H₁ antagonist is 0.001-1% (w/v).

18. The method of claim 17 wherein the pharmaceutically effective amount of the H₁ antagonist is 0.05-0.2% (w/v).

19. The method of claim 1 wherein the pharmaceutically effective amount of the anti-TNFα compound is 0.1-8% (w/v).

20. The method of claim 19 wherein the pharmaceutically effective amount of the anti-TNFα compound is 1-5% (w/v).
